(19) United States
(12) Patent Application Publication
(10) Pub. No.: US 2021/0173143 A1
(43) Pub. Date: Jun. 10, 2021

(54) OPTICAL FIBER FOR TRANSMITTING BOTH AN ILLUMINATION LIGHT AND A LASER LIGHT BEAM

(71) Applicant: Alcon Inc., Fribourg (CH)

(72) Inventors: Chenguang Diao, Irvine, CA (US); Ronald T. Smith, Irvine, CA (US); Alireza Mirsepassi, Irvine, CA (US)

(21) Appl. No.: 17/109,375

(22) Filed: Dec. 2, 2020

Related U.S. Application Data
(60) Provisional application No. 62/943,331, filed on Dec. 4, 2019.

(51) Int. Cl. G02B 6/02

(52) U.S. Cl. CPC G02B 6/02319; A61F 9/008

(57) ABSTRACT

The present disclosure relates to a fiber and a laser probe assembly with a probe tip that houses the fiber. In certain aspects, the fiber includes a core and an outer cladding surrounding the core. The core is configured to transmit a laser light beam while the core and the outer cladding are both configured to transmit an illumination light. Using a fiber that is configured to transmit a laser light beam as well as an illumination light allows for a more compact fiber and probe tip, allowing for medical procedures that require a narrower probe.

### PRIORITY CLAIM

This application claims the benefit of priority of U.S. Provisional Patent Application Ser. No. 62/943,331 titled “OPTICAL FIBER FOR TRANSMITTING BOTH AN ILLUMINATION LIGHT AND A LASER LIGHT BEAM,” filed on Dec. 4, 2019, whose inventors are Chenguang Diao, Ronald T. Smith and Alireza Mirsepassi, which is hereby incorporated by reference in its entirety as though fully and completely set forth herein.

### DESCRIPTION OF THE RELATED ART

In a wide variety of medical procedures, laser light is used to assist the procedure and treat patient anatomy. For example, in laser photocoagulation, a laser probe is used to cauterize blood vessels on the retina. Some probes include a fiber optic cable containing one fiber for delivering laser light to the surgical site, and a separate fiber for delivering illumination light at the same time during an eye surgery procedure, for instance, a bimanual operation. In such cases, one of the two fibers is connected to a laser source to deliver the laser beam, and the other fiber is connected to an illumination source for illumination light, and the two fibers are combined and tightly packed within a tube of the fiber optic cable to minimize the size of the fiber optic cable and, therefore, the size of the probe tip where the fiber optic cable is placed. Using a probe tip with a smaller gauge size is advantageous because it helps with minimizing the size of the incision on the eye (for example, minimum-invasive eye surgery), and helps patients recover faster post-surgery.

However, a fiber optic cable containing a laser fiber as well as an illumination fiber can only be made so narrow, because there must be room for both the illumination fiber and the laser fiber to be placed side-by-side in the tube. Also, narrowing of the two fibers themselves results in lower laser coupling efficiency and insufficient illumination to perform the medical procedure. Further, the fabrication of the probe integrating the two separate fibers (where one fiber is for the laser beam, and the other fiber is for the illumination light), is complicated, and the cost of manufacturing the probe is high. In addition, the thermal robustness of the probe is an issue at high laser power due to the plastic fiber used for illumination light, and the adhesive used to bind the fibers together at the distal end of the probe.

### SUMMARY

According to one embodiment, a laser probe assembly is provided, including a probe body shaped and sized for grasping by a user, and a probe tip housing a fiber. The fiber includes a core, an outer cladding surrounding the core. The core is configured to transmit a laser light beam. The outer cladding is configured to transmit an illumination light.

According to another embodiment, a fiber is provided, including a core and an outer cladding surrounding the core. The core is configured to transmit a laser light beam. The outer cladding is configured to transmit the illumination light.

According to yet another embodiment, a surgical laser system is provided, including an illumination light source configured to emit an illumination light onto a focusing lens, a laser light source configured to emit a laser light beam onto the focusing lens, and the focusing lens. The focusing lens is configured to focus the illumination light onto a core and an outer cladding of a fiber coupled to the surgical laser system and focus the laser light beam onto the core of the fiber, wherein the fiber is downstream from the focusing lens. The fiber comprises the core configured to transmit the illumination light and the laser light beam and the outer cladding surrounding the core, wherein the outer cladding is configured to transmit the illumination light.

### BRIEF DESCRIPTION OF THE DRAWINGS

For a more complete understanding of the present technology, its features, and its advantages, reference is made to the following description, taken in conjunction with the accompanying drawings, in which:

FIG. 1A illustrates a plan view of a system for generating laser light beams for delivery to a surgical target, in accordance with a particular embodiment of the present disclosure.

FIG. 1B illustrates a plan view of a surgical laser system, in accordance with a particular embodiment of the present disclosure.

FIG. 2 illustrates a plan view of a probe, in accordance with a particular embodiment of the present disclosure.

FIGS. 3A-3B illustrate a fiber, in accordance with a particular embodiment of the present disclosure

FIG. 4 illustrates a portion of a fiber, in accordance with a particular embodiment of the present disclosure.

FIG. 5 illustrates a portion of a fiber with an inner cladding, in accordance with a particular embodiment of the present disclosure.

FIG. 6 illustrates a partial cross-sectional view of a probe tip and a fiber, in accordance with a particular embodiment of the present disclosure.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described example is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Embodiments of the disclosure generally relate to fibers and laser probe assemblies. A fiber includes a core that transmits a laser light beam, and the core and an outer cladding surrounding the core that transmits illumination light. A laser probe assembly includes a fiber, and the laser probe assembly allows the user to direct a laser light beam and illumination light in a single fiber. The combination of the transmission of laser light and illumination light in the same fiber results in a more compact fiber optic cable, allowing for medical procedures that require a narrower probe. Embodiments of the disclosure may be especially useful for, but are not limited to, a fiber that can transmit both laser light and illumination light.

As used herein, the term “about” may refer to a +/−10% variation from the nominal value. It is to be understood that such a variation can be included in any value provided herein.

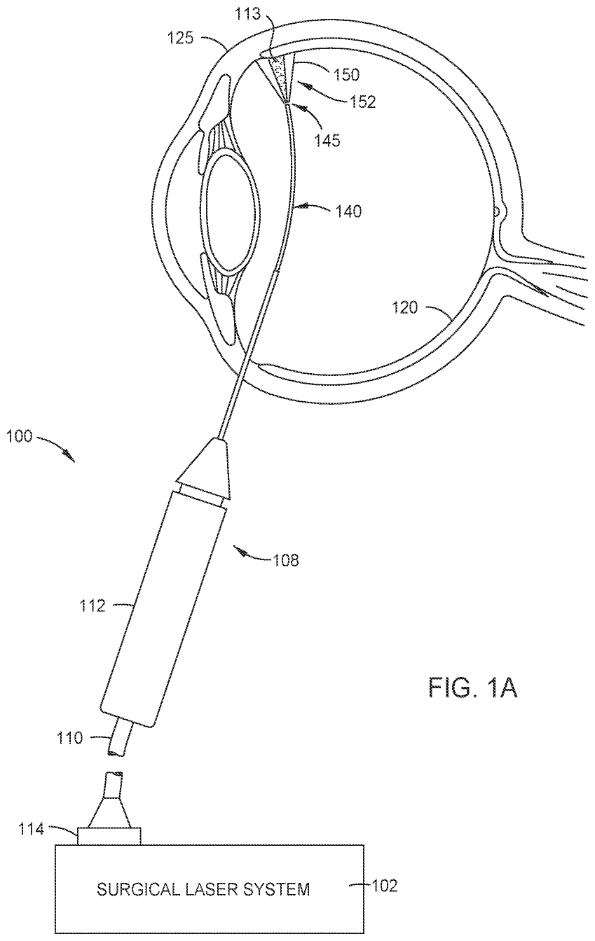

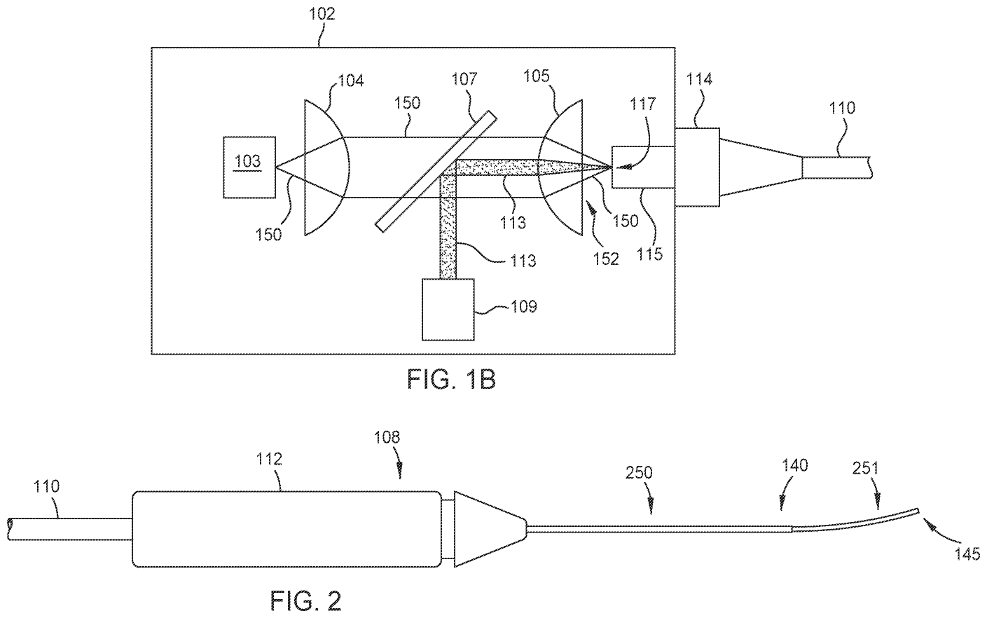

FIG. 1A illustrates a plan view of a system 100 for generating an illumination beam as well as a laser light beam for delivery to a surgical target, in accordance with a particular embodiment of the present disclosure. As shown, system 100 includes a surgical laser system 102 and a probe 108. The system 100 produces an illumination beam 150 and a laser light beam 113 to be delivered to the retina 120 of a patient's eye 125, in one example.

The surgical laser system 102 includes a number of laser light sources (e.g., one or more laser light sources) for generating laser light beams that can be used during an ophthalmic procedure. The surgical laser system 102 may be an ophthalmic surgical laser system configured to generate a laser light beam (e.g., a surgical treatment beam). A user, such as a surgeon or surgical staff member, can control the surgical laser system 102 (e.g., via a foot switch, voice commands, etc.) to fire the laser light beam to treat patient anatomy, e.g., perform photocoagulation. In some instances, the surgical laser system 102 includes a port, and the illumination beam and the laser light beam can be emitted through the port in the surgical laser system 102.

System 100 can deliver the laser light beam 113 and the illumination light 150 from the port to a probe 108 via a fiber contained in the fiber optic cable 110. As shown, probe 108 includes a probe body 112, a probe tip 140, and a distal end 145 of the probe tip. In operation, a laser light source of surgical laser system 102 generates the laser light beam 113, while an illumination light source generates the illumination light 150. The surgical laser system 102 multiplexes the laser light beam 113 and the illumination light 150 into a multiplexed beam 152. The multiplexed beam 152 is directed to a lens of the surgical laser system 102 to focus the multiplexed beam onto an interface plane of a proximal end of the fiber within the fiber optic cable 110, such that the multiplexed beam is transmitted along an entire length of the fiber. The interface plane of the proximal end of the fiber is exposed by a ferrule inserted into a port adapter 114 through which fiber optic cable 110 connects to the surgical laser system 102.

The multiplexed beam 152 is transmitted by the fiber to the probe 108 disposed at the distal end of the fiber optic cable 110. The multiplexed 152 beam exits the probe tip 145 and is projected onto the retina 120. Thus, the surgical laser system 102 is configured to deliver the multiplexed beam 152 to the retina 120 through the fiber of the fiber optic cable 110. The multiplexed beam 152 includes both the laser light beam 113 for the surgical procedure and illumination light 150 to aid the user in the procedure, although the beam associated with the laser light beam is narrower.

Note that, herein, a distal end of a component refers to the end that is closer to a patient's body, or where the laser light beam is emitted out of the laser probe 112. On the other hand, the proximal end of the component refers to the end that is facing away from the patient's body or in proximity to, for example, the surgical laser source 102.

FIG. 1B illustrates a plan view of a surgical laser system 102, in accordance with a particular embodiment of the present disclosure. As shown, the surgical laser system 102 includes a first lens 104 (e.g., collimating lens), a beam splitter 107, a fiber optic cable 110, a second lens 105 (e.g., focusing lens), an illumination light source 103, and a laser light source 109. The beam splitter 107 is downstream from the first lens 104, the second lens 105 is downstream from the beam splitter 107, and the fiber optic cable 110 is downstream from the second lens 105. The illumination light source 103 emits an illumination light 150. The illumination light 150 can be any spectrum of light, including, but not limited to, visible light or white light. The illumination light source 103 can be a light-emitting diode (LED) or a broadband laser source. The illumination light 150 is collimated by the first lens 104 such that the illumination light 150 is transformed into a beam of light with parallel rays, as shown. The first lens 104 can be any lens, including a plano-convex or biconvex lens. The beam splitter 107 allows the illumination light 150 to pass through the beam splitter 107 with a small fraction of the light reflected off the beam splitter. The illumination light 150 is then focused by the second lens 105, as shown. The second lens 105 can be any lens used to focus light, including a plano-convex or biconvex lens. The illumination light 150 and laser beam 113 are focused and incident on the fiber optic cable 110 as a multiplexed beam 152, which is described in greater detail below.

The second lens 105 focuses the multiplexed beam 152 into an interface plane of a proximal end of a fiber that is contained within the fiber optic cable 110. As shown, fiber optic cable 110 is coupled to the surgical laser system 102 through port adapter 114, which receives a ferrule 115 that exposes an interface plane of the proximal end of the fiber that is contained within fiber optic cable 110. More specifically, the interface plane of the proximal end of the fiber is exposed through an opening 117 of ferrule 115. The second lens 105 focuses multiplexed beam 152 onto an interface plane of the proximal end of the fiber such that the multiplexed beam is propagated through the fiber to the distal end of a surgical probe (e.g., probe 108 of FIG. 1) that is coupled to cable 110.

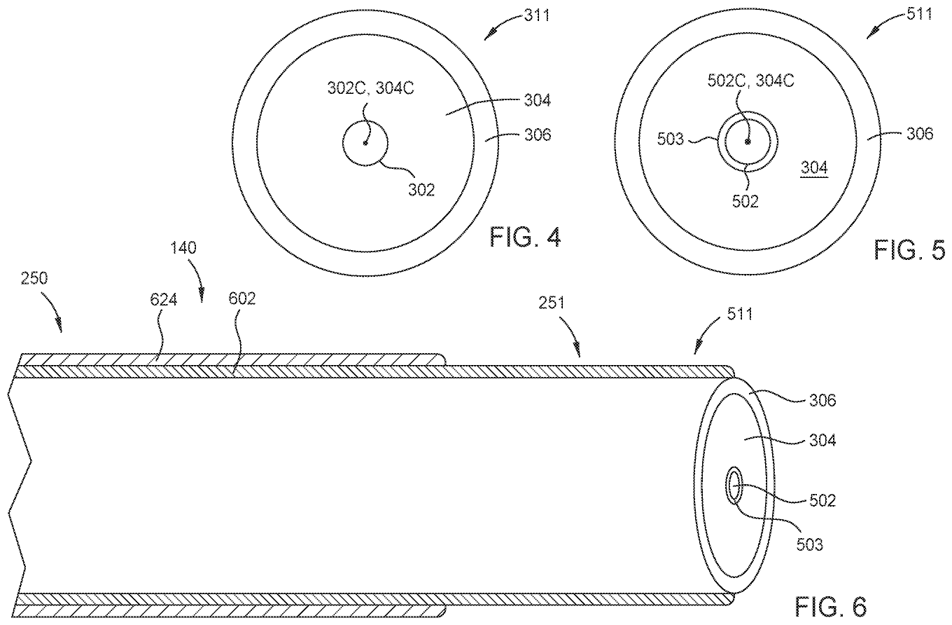

The fiber optic cable 110 may include a fiber (e.g., fiber 300, a portion 311 of which is shown in FIG. 4) having a core and an outer cladding, in some embodiments. In such embodiments, the second lens 105 is configured to focus the illumination light 150 onto both the core and the outer cladding, in which case both the outer cladding and the core transmit the illumination light 150.

In yet some other embodiments, fiber optic cable 110 may include a fiber (e.g., the fiber whose portion 511 is shown in FIG. 5) having a core, an inner cladding, and an outer cladding. In such embodiments, the illumination light 150 is focused on the core, the inner cladding, and the outer cladding in which case the core, the inner cladding, and outer cladding all transmit the illumination light 150.

A laser light source 109 emits a laser light beam 113. The laser light beam 113 can have any desired wavelength, such as from about 532 nm to about 635 nm. The laser light source 109 can emit a variety of wavelengths desired by the user. The laser light beam 113 is reflected by the beam splitter 107 onto focusing lens 105. The laser light beam 113 is then focused by the second lens 105 onto an interface plane of the proximal end of fiber optic cable 110, as part of the multiplexed beam 152. The laser light beam 113 is transmitted by the core of the fiber optic cable 110. The surgical laser system 102 provides both the illumination light 150 and the laser light beam 113 to the fiber optic cable 110 as the multiplexed beam 152. Thus, a single fiber in the fiber optic cable 110, including a core and an outer cladding, is capable of transmitting both the laser light beam 113 (through the core) and illumination light 150 (through the outer cladding and the core) in the same fiber.

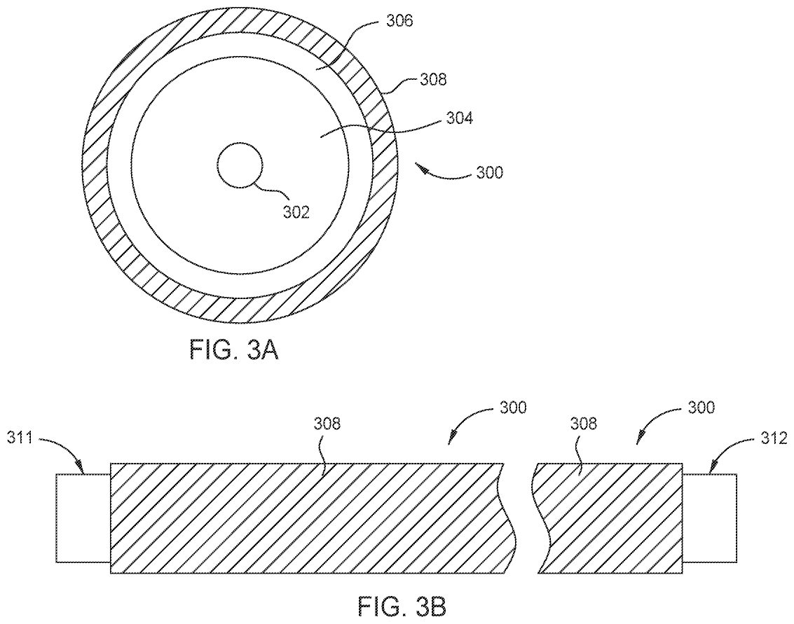

FIG. 2 illustrates a plan view of the probe 108, in accordance with a particular embodiment of the present disclosure. As described above, the probe 108 includes a probe body 112 shaped and sized for grasping by a user. Extending from the probe body 112 is the probe tip 140 with a distal end 145. The fiber optic cable 110 typically comprises a fiber (e.g., fiber 300 of FIG. 3, the fiber of FIG. 5, etc.) surrounded by a polyvinyl chloride (PVC) tube for protecting the fiber during handling. The fiber extends through the probe body 112 and into the probe tip 140. The multiplexed beam 152 emanates from the distal end of the fiber and, thereby, the distal end 145 of the probe tip 140 onto the retina. In some embodiments, the probe tip 140 comprises a first straight portion 250 and a second curved portion 251. The first straight portion 250 includes a sleeve of the probe tip, and the second curved portion 251 includes a tube surrounding the fiber. The embodiment of FIG. 2 is merely shown as an example. In other examples, a probe tip may be straight throughout, or the sleeve 250 is not included. A variety of other configurations are also possible and are not outside the scope of this disclosure, as one of ordinary skill in the art can appreciate.

FIGS. 3A-B illustrate a fiber 300, in accordance with a particular embodiment of the present disclosure. As shown, the fiber 300 includes a core 302, an outer cladding 304, a coating 306, and a buffer 308. The buffer 308 can include plastic, such as ethylene tetrafluoroethylene (ETFE). The buffer 308 is stripped at the proximal end of the fiber 300 so that a proximal end portion 311 (“portion 311”) of the fiber can be inserted to the ferrule. The buffer is also stripped at the distal end of the fiber 300 so that a distal end portion 312 (“portion 312”) of the fiber 300 can be inserted into probe tip 140, according to some embodiments.

FIG. 4 illustrates a front view of the portion 311 of fiber 300, in accordance with a particular embodiment of the present disclosure. The portion 311 includes a core 302 disposed in an outer cladding 304, and the outer cladding 304 includes a material that can include fused silica. Note, however, that the portion 311 does not include the buffer 308, as the buffer 308 has been stripped from around the portion 311. FIG. 4 can also illustrate a front view of the portion 312 of fiber 300, which also does not include the buffer 308. Laser light beam provided by a laser light source of the surgical laser system 102 is directed into the core 302 of the fiber 300. Thus, the core 302 conducts the laser light beam along the length of the fiber 300. Both core 302 and outer cladding 304 may include fused silica. However, the core 302 is doped with a dopant that increases the index of refraction of the core 302. Therefore, the refractive index of the core 302 is greater than the refractive index of the outer cladding 304, such that the laser light beam traveling along the core 302 is contained within the core and prevented from escaping from the core 302 into the outer cladding 304. In one example, the dopant can include germanium (Ge). The core 302 and the outer cladding 304 transmit illumination light from the surgical laser system 102. Thus, a single fiber including the core 302 and the outer cladding 304 is capable of transmitting both the laser light beam (through the core 302) and illumination light (through the outer cladding 304 and the core 302). In addition, using fused silica for transmitting the illumination light, such as in fiber 300 of FIG. 3 or the fiber of FIG. 5, results in a more thermally stable fiber as compared to a conventional illumination fiber that is made of traditional plastic, and there is no need to use adhesive to bond two fibers, which makes the fiber more thermally robust.

A coating 306 is formed over the outer cladding 304. In some instances, the coating 306 is a hard polymer coating. In other instances, the coating 306 is formed from other materials, such as acrylate. The refractive index of the coating 306 is less than the refractive index of the outer cladding 304, such that the illumination light traveling along the outer cladding 304 is contained within the outer cladding 304 and prevented from escaping from the outer cladding 304 into the coating 306. In certain embodiments, the numerical aperture (NA) between the outer cladding 304 and the coating 306 is greater than about 0.5 to provide the wide illumination required in some surgical cases.

FIG. 5 illustrates a portion 511 of a fiber with an inner cladding 503, in accordance with a particular embodiment of the present disclosure. Portion 511 corresponds to a proximal or a distal portion of a fiber, where the fiber's buffer has been stripped. In the fiber of FIG. 5, the inner cladding 503 surrounds a core 502 and the outer cladding 304 surrounds the inner cladding 503. The inner cladding 503 can include fused silica doped with dopants, the dopants including fluorine, chlorine, boron, or any combination of the above, according to some embodiments. The dopants change the optical properties of the inner cladding 503, for example, the refractive index. In certain embodiments, the NA between the core 502 and the inner cladding 503 is from about 0.20 to about 0.30, such as about 0.22. The inner cladding 503 keeps the laser light beam from entering the outer cladding 304 by causing partial or total internal reflection of the laser light beam, thus keeping the laser light beam in the core. As described above, in the example of FIG. 5, the illumination light is focused by the surgical laser system onto core 502, inner cladding 503 and outer cladding 304 while the laser light beam is focused on core 502.

Referring to FIGS. 4 and 5, in certain embodiments, the diameter of the core 302, 502 is from about 70 μm to about 80 μm, the outer diameter of the outer cladding 304 is from about 290 μm to about 300 μm, and the outer diameter of the coating 306 is from about 320 μm to about 330 μm. The location of the center 302c, 502c of the core 302, 502 is approximately the same location as the center 304c of the outer cladding 304, according to one embodiment. Other diameters are also contemplated.

FIG. 6 illustrates a partial cross-sectional view of a probe tip 140, in accordance with a particular embodiment of the present disclosure. The portion 511 is surrounded by the tube 602, and the tube is surrounded by the sleeve 624 of the probe tip 140. The tube 602 can include any suitable material, e.g., Nitinol, nickel titanium, or stainless steel. The sleeve 624 can include, for example, stainless steel. In the example of FIG. 6, the distal end of the portion 511 and the distal end of the tube 602 surrounding the fiber extend beyond the distal end of the sleeve 624 of the probe tip 140. Thus, the first straight portion 250 of the probe tip 140 includes the sleeve 624, whereas the second curved portion 251 of the probe tip does not include the sleeve, although the portion 511 is still surrounded by the tube 604 in the second curved portion. In other embodiments, the sleeve 624 extends to cover the entire portion 511 throughout the probe tip 140. In other embodiments, the probe tip 140 includes the tube 602 and the sleeve 624 is not included. Although the portion 511 illustrated in FIG. 6 includes the inner cladding 503, the fiber optic cable could instead include the portion 311 (which does not include the inner cladding), without any loss of generality. As described above, the embodiment of FIG. 6 is merely shown as an example. One of ordinary skill in the art can appreciate other embodiments with different configurations (e.g., a completely straight probe tip, or a probe tip with a distal end that is flush with the distal ends of the fiber of FIG. 5 and tube 602) which are also not outside the scope of this disclosure.

As described above, a fiber optic cable is capable of transmitting both a laser light beam through a core, and illumination light through the core and an outer cladding. The fiber optic cable does not have two separate fibers for illumination light and the laser light beam, but rather one fiber that includes a core to transmit the laser light beam, and the core and an outer cladding to transmit the illumination light. The fiber optic cable can be used in a system for medical procedures, and the system provides both laser light beam for the cauterizing or burning, and illumination light to aid the user in performance of the procedure.

The use of a combined core and outer cladding to transmit both the laser light beam and illumination light results in a more compact fiber, and removes the need for adhering two fibers together. The narrower fiber is useful for medical procedures that require thinner probe tips. In addition, the fiber optic cable is more thermally stable than a traditional fiber optic cable, due to the lack of thermally unstable adhesive. The use of a single fiber in the fiber optic cable removes the need for two connectors (one for each fiber), and thus only one connector is necessary, which reduces the manufacturing and labor costs, as there is no need to handle assembly of two fibers

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A laser probe assembly, comprising:
a probe body shaped and sized for grasping by a user; and
a probe tip housing a fiber, the fiber comprising:
a core configured to transmit a laser light beam; and
an outer cladding surrounding the core, wherein the outer cladding is configured to transmit an illumination light.

2. The laser probe assembly of claim 1, wherein:
the fiber further comprises an inner cladding;
the inner cladding surrounds the core;
the outer cladding surrounds the inner cladding; and
wherein the inner cladding is also configured to transmit the illumination light.

3. The laser probe assembly of claim 2, wherein:
the core and the outer claddings comprise fused silica; and
the inner cladding comprises fluorine-doped fused silica.

4. The laser probe assembly of claim 1, wherein the fiber further comprises a coating surrounding the outer cladding.

5. The laser probe assembly of claim 4, wherein the coating has a refractive index that is less than a refractive index of the outer cladding.

6. The laser probe assembly of claim 1, wherein the core is doped with a dopant such that a refractive index of the core is greater than a refractive index of the outer cladding.

7. The laser probe assembly of claim 7, wherein the dopant comprises germanium (Ge).

8. The laser probe assembly of claim 1, wherein:
the illumination light and the laser light beam are provided by a surgical laser system coupled to a proximal end of the fiber;
the illumination light is focused by a lens of the surgical laser system onto the core and the outer cladding; and
the laser light beam is focused by the lens of the surgical laser system onto the core.

9. The laser probe assembly of claim 1, wherein the core is further configured to transmit the illumination light.

10. A fiber, comprising:
a core configured to transmit a laser light beam; and
an outer cladding surrounding the core, the outer cladding configured to transmit the illumination light.

11. The fiber of claim 10, wherein the core is further configured to transmit the illumination light.

12. The fiber of claim 10, wherein the core comprises fused silica, and wherein the outer cladding comprises fused silica.

13. The fiber of claim 11, wherein the fiber further comprises an inner cladding, the inner cladding surrounding the core, the outer cladding surrounding the inner cladding, and wherein the inner cladding is also configured to transmit the illumination light.

14. The fiber of claim 13, wherein the inner cladding comprises fluorine-doped fused silica.

15. The fiber of claim 10, wherein the core is doped with a dopant such that a refractive index of the core is greater than a refractive index of the outer cladding.

16. The fiber of claim 15, wherein the dopant comprises germanium (Ge).

17. The fiber of claim 10, wherein:
the illumination light and the laser light beam are provided by a surgical laser system coupled to a proximal end of the fiber;
the illumination light is focused by a lens of the surgical laser system onto the core and the outer cladding; and
the laser light beam is focused by the lens of the surgical laser system onto the core.

18. The fiber of claim 10, the fiber further comprises a coating surrounding the outer cladding, the coating having a refractive index that is less than a refractive index of the outer cladding.

19. A surgical laser system, comprising:
an illumination light source configured to emit an illumination light onto a focusing lens;
a laser light source configured to emit a laser light beam onto the focusing lens;
the focusing lens configured to:
focus the illumination light onto a core and an outer cladding of a fiber coupled to the surgical laser system; and
focus the laser light beam onto the core of the fiber, wherein the fiber is downstream from the focusing lens, the fiber comprising:
the core configured to transmit the illumination light and the laser light beam; and
the outer cladding surrounding the core, wherein the outer cladding is configured to transmit the illumination light.

20. The surgical laser system of claim 19, wherein:
the fiber further comprises an inner cladding, the inner cladding surrounding the core, the outer cladding surrounding the inner cladding;
the focusing lens is configured to focus the illumination light onto the inner cladding; and
the inner cladding is configured to transmit the illumination light.
